(19) United States
(12) Patent Application Publication
(10) Pub. No.: US 2006/0122227 A1
(43) Pub. Date: Jun. 8, 2006

(54) Process for alkylating secondary amines and the use in donepezil preparation thereof

(76) Inventors: Lior Zelikovitch, Mazkeret Batia (IL); Oded Arad, Rechovot (IL); Mohammed Alnabari, Hura (IL); Yana Sery, Beer-Sheva (IL); Orna Kurlat, Beer-Sheba (IL); Moshe Bentolila, Moshav Tkuma (IL); Aric Abadayev, Beer-Sheba (IL); Hanit Marom, Haifa (IL); Hila Isenberg, Beer-Sheba (IL); Joseph Kaspi, Givatayim (IL)

Correspondence Address: Martin MOYNIHAN;PRTSI, Inc., P.O. Box 16446, ARLINGTON, VA, 22215, US

(21) Appl. No.: 11/235,105

(22) Filed: Sep. 27, 2005

Related U.S. Application Data
(60) Provisional application No. 60/613,706, filed on Sep. 29, 2004.

(51) Int. Cl. C07D 211/06; A61K 31/445

(52) U.S. Cl. 514/319; 546/206

(57) ABSTRACT

The present invention relates to an improved process of alkylating secondary amines, more particularly of alkylating compounds having amino piperidinic group, which are useful as donepezil intermediates, wherein an alcohol serves as reaction facilitator thus enabling to obtain donepezil and salts thereof in high quality and yield. The present invention also relates to the prevention of unwanted alkylation of donepezil precursors, having amino piperidinic group, by using suitable reaction conditions.

### RELATED APPLICATIONS

The present application claims the benefit of U.S. Provisional Patent Application No. 60/613,706, filed on Sep. 29, 2004, which is incorporated herein by reference.

### FIELD OF THE INVENTION

The present invention relates to alkylation of secondary amines, more particularly to alkylation of compounds having amino piperidinic group, which are useful as donepezil intermediates.

### BACKGROUND OF THE INVENTION

Donepezil hydrochloride (I) is a reversible acetylcholinesterase inhibitor that has the following structure: 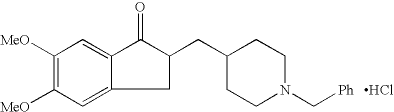
donepezil hydrochloride (I)

Donepezil (known as 1-benzyl-4-[(5,6-dimethoxy-1-indanon-2-yl)methyl]piperidine) hydrochloride is an effective drug for treating dementia and Alzheimer's disease. The drug is administrated in the form of oral solid formulations such as 5 and 10 mg film coated tablets, capsules and granules and is given to the patients once daily.

The preparation of donepezil hydrochloride was first described in the European Patent Number EP 296560.

By reacting 5,6-dimethoxy-1-indanone with 1-benzyl-4-formylpiperidine in the presence of a strong base such as lithium diisopropylamide, the product 1-benzyl-4-[(5,6-dimethoxy-1-indanon)-2-ylidenyl]methylpiperidine hydrochloride is obtained. The next step is catalytic hydrogenation with 10% palladium on charcoal in THF. The product is purified by column chromatography using silica gel eluting with dichloromethane: methanol mixture (50:1). The process is illustrated in scheme 1.

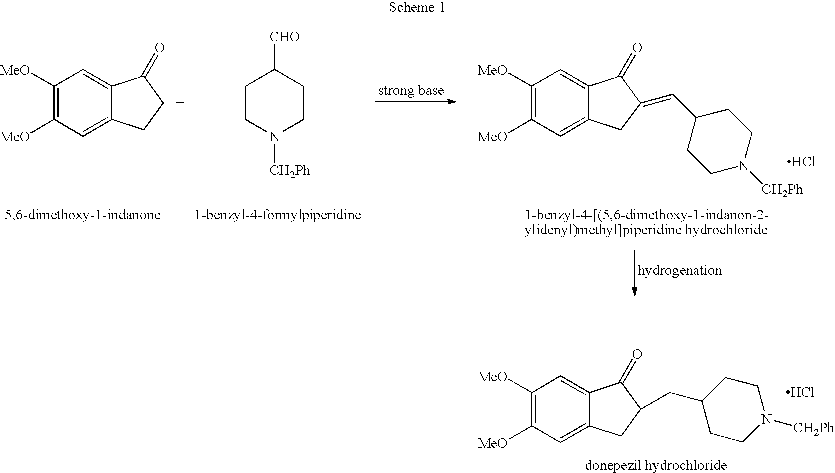

The process described in the European Patent Number EP 296560 suffers from several drawbacks. The first is concerned with using the base lithium diisopropylamide, which is produced in situ by dissolving diisopropylamine in THF followed by addition of n-butyl lithium in hexane, the latter reagent being corrosive and highly flammable material. Another problem is the reaction temperature, which is very low (−78° C.) hence the reaction is problematic with respect to industrial scaling up. The third problem is associated with using column chromatography, which is not preferred for industrial application.

In order to overcome the disadvantages of the above mentioned process, we have developed an alternative process for preparing donepezil, which is provided in European Patent Application Number 1386607 (to the present applicant). The process is described in scheme 2.

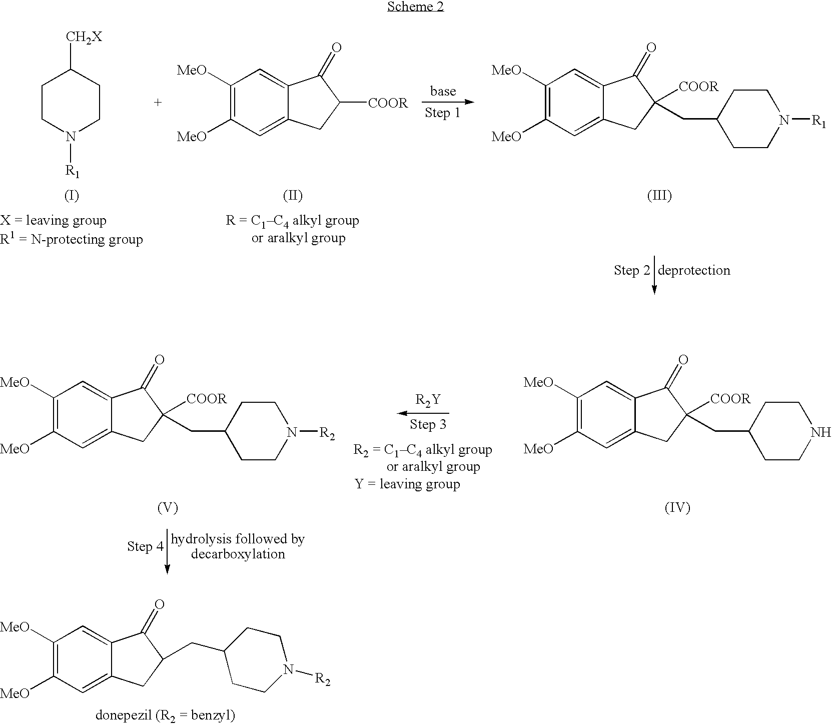

The process which is provided in European Patent Application Number 1386607 comprises the following steps:

1. Reacting N-protected activated-4-methylpiperidine (I), wherein X represents a leaving group such as halide, mesylate or tosylate and R₁ represents an N-protecting group such as t-butoxycarbonyl (t-BOC), benzyloxycarbonyl (CBZ), and triphenylmethyl, with 2-alkoxycarbonyl-5,6-dimethoxyindan-1-one, wherein R represents a C₁-C₄ alkyl group such as methyl, ethyl, t-butyl or an aralkyl group such as (optionally substituted) benzyl group thus affording 4-[(2-alkoxycarbonyl-5,6-dimethoxy-indan-1-on-2-yl)methyl]-N-protected-piperidine (III).

An example of 2-alkoxycarbonyl-5,6-dimethoxyindan-1-one (II) is the compound 5,6-dimethoxy-2-ethoxycarbonylindan-1-one, which is prepared by reacting 5,6-dimethoxyindan-1-one with diethyl carbonate in THF in the presence of sodium hydride under reflux. An example of N-protected activated-4-methyl-piperidine (I) is N-CBZ-4-iodomethylpiperidine, which is prepared from CBZ-piperidinemethanol.

An example of the product (III) in this case is: 1-CBZ-[4-(5,6-dimethoxy 2-ethoxycarbonyl-indan-1-on-2-yl)methyl]piperidine.

2. Deprotecting compound (III) so as to afford 4-[(2-alkoxycarbonyl-5,6 dimethoxy indan-1-on-2-yl)methyl]piperidine having the formula (IV).

An example of the compound (IV) is: [4-(5,6-dimethoxy-2-ethoxycarbonyl-indan-1-on-2-yl)methyl]piperidine, which may be obtained from compound (III) by catalytic hydrogenation with 10% palladium on charcoal in ethanol.

3. Reacting the compound of the type (IV) with a compound of the formula R₂Y, wherein R₂ is C₁-C₄ alkyl group or an aralkyl group and Y is a leaving group thus affording a compound of the type (V).

An example for a compound of the formula R₂Y is benzyl chloride, hence the compound of the type (V) is 1-benzyl-[4-(5,6-dimethoxy-2-ethoxycarbonyl-indan-1-on-2-yl)methyl]piperidine, which may be obtained by reacting [4-(5,6-dimethoxy-2-ethoxycarbonyl-indan-1-on-2-yl)methyl]piperidine with benzyl chloride in toluene with triethylamine or as will be detailed herein.

4. Compound of the type (V) is subjected to hydrolysis followed by decarboxylation to afford donepezil, wherein R₂ is a benzyl group. The reaction may be carried out by dissolving the compound in ethanol:water mixture and reacting with sodium hydroxide to afford donepezil base, which may be crystallized from ethanol.

The novel compounds represented by the general formulae (III), (IV) and (V) were isolated and identified as intermediates in the process, as provided in EP Patent Application No.1386607.

In the U.S. patent application, entitled “Use of purified donepezil maleate for preparing pharmaceutically pure amorphous donepezil hydrochloride”, by the present inventors, which claims priority from U.S. Provisional Patent Application No. 60/613,707, which is filed concurrently with the present application and which is incorporated by reference as if fully set forth herein, a novel process for donepezil hydrochloride preparation via purified donepezil maleate is disclosed. In this process, the reaction mixture is treated with aqueous solution of maleic acid, thus a maleate salt precipitates selectively as donepezil maleate. The salt is insoluble neither in the aqueous medium nor in the organic solvent and therefore the precipitation is selective in addition to being almost quantitative, while most of the impurities are left either in the aqueous phase or in the organic phase.

The crude donepezil maleate may be isolated and consequently crystallized. Optionally, the purified donepezil maleate may be suspended in a mixture of toluene and water. Aqueous NaOH is added and the phases are separated (donepezil base is dissolved in toluene). Donepezil base solution is washed with water and the phases are separated. Equimolar quantity of aqueous hydrochloric acid solution is added, and phases are separated (donepezil HCl is dissolved in water). An inactive pharmaceutical ingredient is optionally added, solution is stirred and freeze-dried. Thus donepezil maleate may be readily converted to donepezil hydrochloride without formation of any impurities and consequently used in pharmaceutical dosage forms.

While scaling-up the process of obtaining donepezil it has been surprisingly found that the alkylation step, of intermediates having amino piperidinic group, may be substantially improved by adding an alcohol to the reaction mixture containing an alkylating agent such as benzyl chloride and a base in an organic solvet.

### SUMMARY OF THE INVENTION

The present invention relates to an improved process of alkylating secondary amines, more particularly to a process of alkylating compounds having amino piperidinic group, which are useful as donepezil intermediates, wherein an alcohol serves as reaction facilitator thus enabling to obtain donepezil or salts thereof in high quality and yield.

An exemplary alkylation process is the reaction of [4-(5,6-dimethoxy-2-ethoxycarbonyl-indan-1-on-2-yl)methyl]piperidine with benzyl bromide to obtain 1-benzyl-[4-(5,6-dimethoxy-2-ethoxycarbonyl-indan-1-on-2- yl)-methyl]piperidine.

The present invention also relates to an improved halogenation process wherein unwanted alkylation of the donepezil precursors, having amino piperidinic group, is prevented by using suitable reaction conditions

### DETAILED DESCRIPTION OF THE INVENTION

The present invention provides an improved process for alkylating secondary amines, more specifically for alkylating intermediates having an amino piperidinic group using an alkylating agent, wherein the reaction is catalyzed by addition of an alcohol, which assists in bringing the reaction to completion.

The alkylation is described in step 3 of scheme 2 above, wherein the compound of the type (IV) is reacted with the compound of the formula R₂Y, while R₂ represents C₁-C₄ alkyl group or an aralkyl group and Y is a leaving group, (e.g. Y═Cl, Br, and I), preferably R₂=PhCH₂ (benzyl). Typically R₂Y is an alkylating agent selected from the group consisting of benzyl bromide, benzyl chloride, and benzyl iodide, preferably benzyl chloride.

In a preferred embodiment, the present invention provides a process of alkylating the compound of the type (IV), containing the amino piperidinic group, comprising:
a) dissolving the compound of the type (IV), containing the amino piperidinic group, in an organic solvent and adding a base;
b) optionally adding an alcohol;
c) heating the reaction mixture, adding an alkylating agent optionally drop-wise and stirring for a time period sufficient to allow completing the reaction; and
d) isolating the product.

The product of the reaction is a compound of the type (V) such as 1-benzyl-[4-(5,6-dimethoxy-2-ethoxycarbonyl-indan-1-on-2-yl)methyl]piperidine.

According to one aspect of the present invention the reaction solvent is selected from the group consisting of ethyl acetate, isopropyl acetate, n-butyl acetate, isobutyl acetate, dichloromethane, chloroform, diethyl ether, diisopropyl ether, methyl tert-butyl ether, toluene, xylenes, and mixtures thereof. Preferably the solvent is toluene.

According to another aspect of the present invention the preferred base is a solid inorganic base selected from the group consisting of sodium hydroxide, potassium hydroxide, sodium carbonate, sodium bicarbonate, potassium carbonate, potassium bicarbonate, and combinations thereof, preferably potassium carbonate.

According to another aspect of the present invention the alkylation can be completed only if a certain amount of an alcohol is added to the reaction mixture. The alcohol is selected from the group consisting of methanol, ethanol, 1-propanol, 2-propanol, 1-butanol 2-butanol, isobutanol, and mixtures thereof, preferably ethanol.

While not wishing to be bound by any particular theory, it is assumed that the alcohol serves as a reaction facilitator in the heterogenic reaction mixture containing the organic hydrophobic solvent and the inorganic solid base, by increasing the solubility of the inorganic reactant in the organic medium, thus improving the solvation of the base in the organic medium. It is further assumed that the alcohol improves the reactivity at the surface of the inorganic base molecule thus assisting to bring the heterogenic reaction to completion.

The present invention is therefore predicated on the surprising and unexpected discovery that the alkylation is completed only if certain amount of an alcohol is added to the reaction mixture. It was found by the inventors of the present invention that the optimal amount of the alcohol was 1-3% v/v relative to the volume of the solvent, preferably 2.5%.

According to the present invention the advantage of using an alcohol in comparison to using a phase transfer catalyst such as tetrabutyl ammonium bromide is that the alcohol is volatile and therefore it may be completely eliminated by evaporation. In contrast while using the phase transfer catalyst there is the risk that a residual impurity consisting of the phase transfer catalyst will be present in the final drug product.

A non-limiting example is the process for obtaining the intermediate 1-benzyl-4-[(5,6-dimethoxy-2-ethoxycarbony-lindan-1-on-2-yl)methyl]piperidine in high quality and yield, which leads to improving the subsequent process steps for obtaining pure donepezil and donepezil salts thereof.

According to another aspect of the present invention, when the reaction is performed without adding an alcohol, it cannot be completed so that the reaction mixture contains substantial quantity of un-reacted starting material, although an excess of benzyl chloride is used in the reaction.

Table 1 and 2 provide results demonstrating the correlation between the kinetic behavior of the alkylation and ethanol amount in the reaction mixture.

More specifically table 1 shows the correlation between the quantity of ethanol and the percentage of the starting material (the compound of the type (IV)) in the alkylation reaction mixture after 9 reaction hours.

TABLE 1

| | % of the starting material of | |
|---|---|---|
| Experiment | type (IV) * in the reaction | % ethanol in the |
| No. | mixture (by HPLC) | reaction solution V/V |
| 1 | 11.4 | 1.2 |
| 2 | 9.7 | 1.5 |
| 3 | 4.5 | 2.0 |
| 4 | 0.3 | 2.5 |
| 5 | 0.4 | 2.6 |
| 6 | 7.2 | 3.0 |
| 7 | 23.0 | 0 |

* The compound of the type (IV) is: 4-[5,6-dimethoxy-2-ethoxycarbonyl-indan-1-on-2-yl)methyl]piperidine.

It may be seen from table I that without adding ethanol, the reaction cannot be completed within a reaction time of 9 hours. The optimal content of ethanol is 2.5%, however if the amount of ethanol is slightly increased the conversion of [4-(5,6-dimethoxy-2-ethoxycarbonyl-indan-1-on-2-yl)methyl]piperidine to 1-benzyl-[4-(5,6-dimethoxy-2-ethoxycarbonyl-indan-1-on-2-yl)methyl]piperidine is decreased.

Table 2 shows the correlation between the reaction time and the percentage of the starting material (IV) in the alkylation reaction mixture.

TABLE 2

| | % of starting | % of starting | % of starting | % ethanol |
|---|---|---|---|---|
| | material (IV) in | material (IV) in | material (IV) in | in the |
| Exper- | the reaction | the reaction | the reaction | reaction |
| iment | mixture after 9 | mixture after 6 | mixture after 4 | solution |
| No. | reaction hours | reaction hours | reaction hours | V/V |
| 5 | 0.4 | 3.7 | 8.4 | 2.6 |
| 6 | 7.2 | 22.9 | 32.6 | 3.0 |

Impurities in the final product may stem from the starting materials but may be produced also by unwanted side reactions. It has been discovered by the inventors of the present invention, during the scaling-up of the hydrogenation process, that the impurity 1-formyl-4-[(5,6-dimethoxy-2-ethoxycarbony-lindan-1-on-2-yl)methyl]piperidine was detected in the residue obtained after the ethanol evaporation. The residue was obtained only when high temperature evaporation was carried out at atmospheric pressure. When the evaporation was carried out at reduced pressure and without application of heat, the impurity was not detected.

While not wishing to be bound by any particular theory, it is assumed that the carbon dioxide, evolved in the deprotection reaction, reacts with hydrogen to yield formic acid, thus the deprotected molecule having secondary amino piperidinic group is transformed to a piredinium formate salt. Upon application of heat, the impurity 1-formyl-[4-[(5,6-dimethoxy-2-ethoxycarbony-lindan-1-on-2-yl)methyl]piperidine is formed. A quantitative amount of formic acid (relative to the expected quantity of carbon dioxide) was detected by titration.

The unwanted side reaction described hereinabove is depicted in scheme 3.

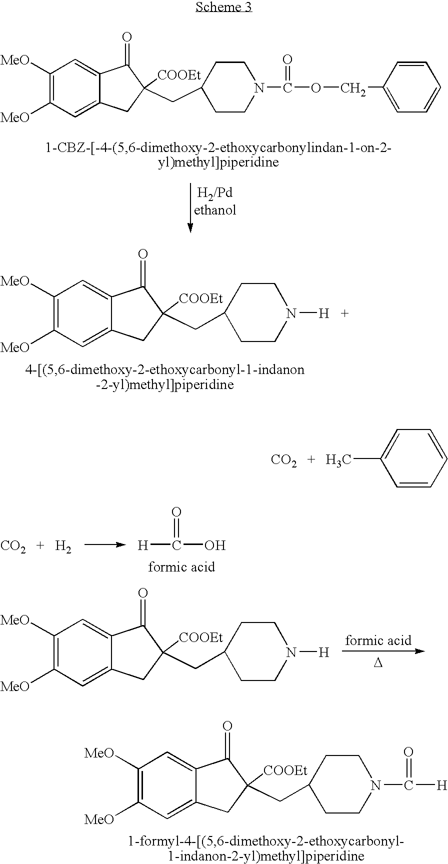

To overcome the problem of the unwanted side reaction described hereinabove the present invention provides also an improved hydrogenation process wherein the unwanted alkylation of donepezil precursors, having amino piperidinic group, is prevented by using suitable reaction conditions

In accordance with the present invention, the improved hydrogenation process comprises the steps of:

a) dissolving 1-CBZ-4-[(5,6-dimethoxy-2-alkoxycarbonylindan-1-on-2-yl)-methyl]piperidine (compound of the type (III)) in an organic solvent and adding a suitable amount of catalyst;

b) hydrogenating optionally at elevated temperature and under pressure;

c) filtering off the catalyst through Celite thus obtaining a solution;

d) evaporating the solvent under reduced pressure and without application of heat, or optionally with cooling, to prevent formation of impurities; and

e) isolating the product (the compound of the type (IV)) as a solid.

According to another aspect of the present invention the compound of the type (IV) is obtained from the compound of the type (III) by catalytic hydrogenation using a catalyst such as 5% or 10% palladium on charcoal in ethanol.

Several compounds of the type (III) may be hydrogenated having the following formula:

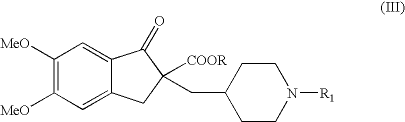

wherein R is a C₁-C₄ alkyl group such as methyl, ethyl, t-butyl or an aralkyl group such as (optionally substituted) benzyl group and R₁ is any appropriate N-protecting group, such as t-butyloxycarbonyl (t-BOC), benzyloxycarbonyl (CBZ), and triphenylmethyl and X is a leaving group, such as halide, mesylate or tosylate.

An exemplary compound of the present invention is CBZ-[4-(5,6-dimethoxy-2-ethoxycarbonyl-indan-1-on-2-yl)methyl]piperidine, which is conveniently hydrogenated in high yield and purity, as described in example 2.

In another aspect of the present invention, the ethanol used in the hydrogenation process should be evaporated to dryness in order not to leave some non-evaporated volume of ethanol in the reaction mixture that might decrease the reaction rate in the next step (the relative volume of ethanol should be less than 3% V/V). It may be seen from table 2, experiment 6, that if the ethanol volume exceeds 3%, relative to reaction volume, the conversion is decreased even at reaction time of 9 hours.

According to another aspect of the present invention, not only higher yields are obtained in the improved hydrogenation process, the purity is also improved. While not wishing to be bound by any particular theory, it is assumed that since the reaction is brought to completion and the starting materials are fully consumed, the content of the starting materials in the reaction mixture after completing the reaction is significantly reduced.

According to another aspect of the present invention the organic solvent used in the hydrogenation process may be selected from the group consisting of THF, methanol, ethanol, 1-propanol, 2-propanol, 1-butanol and 2-butanol, preferably ethanol.

According to another aspect of the present invention the preferable catalyst is 5%-10% palladium on charcoal, more preferably 5% palladium on charcoal, wherein at least 0.8 weight parts of the palladium on charcoal relative to 10 weight parts of the starting material are used in the reaction, and preferably 1 weight part of the palladium on charcoal relative to 10 weight parts of starting material are used in the reaction.

According to another aspect of the present invention the compound 1-formyl [4-[(5,6-dimethoxy-2-ethoxycarbonylindan-1-on-2-yl)methyl]piperidine may be prepared by reacting [4-(5,6-dimethoxy-2-ethoxycarbonylindan-1-on-2-yl)methyl]piperidine and formic acid to obtain the said compound in substantially pure form. A sample of the compound 1-formyl-[4-(5,6-dimethoxy-2-ethoxycarbonylindan-1-on-2-yl)methyl]-piperidine may be used as a reference marker in testing the purity of [4-(5,6-dimethoxy-2-ethoxycarbonylindan-1-on-2-yl)methyl]piperidine, obtained by the hydrogenation of 1-CBZ-[4-(5,6-dimethoxy-2-ethoxycarbonylindan-1-on-2)-ylmethyl]piperidine essentially as described herein.

### EXAMPLES

### Example 1

### Preparation of 1-benzyl-4-[(5,6-dimethoxy-2-ethoxycarbonylindan-1-on-2-yl)methyl]piperidine with addition of ethanol

4-[(5,6-dimethoxy-2-ethoxycarbonylindan-1-on-2-yl)methyl]piperidine (10 g) was dissolved in toluene (100 ml), potassium carbonate was added (10 g) followed by addition of ethanol (2.5 ml). The reaction mixture was heated to 75° C. and benzyl chloride was added drop-wise (4 ml) and stirring was continued for 9 hours. The reaction mixture was cooled to about 55-60° C. and the organic layer was washed twice with water (2×35 ml), then dried over magnesium sulfate and evaporated to obtain 10.6 g of white solid in 85% yield.

### Example 2

### Preparation of 4-[(5,6-dimethoxy-2-ethoxycarbonylindan-1-on-2-yl)methyl]piperidine by catalytic hydrogenation in large scale

Ethanol (16 L) was charged into a cleaned and dry hydrogenator and mixing was applied, the nitrogen pressure was set to about 10 bars. 1-CBZ-4-[(5,6-dimethoxy-2-ethoxycarbonylindan-1-on-2-yl)methyl]piperidine (1.6 Kg) was added and dissolved in ethanol followed by addition of 5% palladium on charcoal (160 g). The nitrogen pressure was set to about 3 bars and the temperature was increased to 60-65° C. The pressure was released and the hydrogenator was washed 3 times with hydrogen until a pressure of 3 bars was achieved. Temperature was increased to 70-75° C. and hydrogen pressure was increased to maximum of 7 bars. The mixture was hydrogenated at temperature of 70-75° C. and maximal pressure of 7 bars for 5 hours. The hydrogenator was cooled to 20-25° C. and the pressure was released. The suspension containing the catalyst was filtered through Celite (400 g), which was washed with additional volume of ethanol (2 L). Ethanol was evaporated without heating to dryness under reduced pressure to obtain 1.108 Kg of the product 4-[((5,6-dimethoxy-2-ethoxycarbonylindan-1-on-2-yl)methyl]piperidine in 95% yield and purity of 98% (by HPLC).

The product may be used in the next step without purification, hence after ethanol evaporation toluene may be added and reaction is continued as described in example 4.

### Example 3

### Large scale preparation of donepezil maleate via 1-benzyl-4-[(5,6-dimethoxy-2-ethoxycarbonylindan-1-on-2-yl)methyl]piperidine

A solution of about 1.1 Kg of 4-[(5,6-dimethoxy-2-ethoxycarbonylindan-1-on-2-yl)methyl]piperidine in toluene (11 L) was charged into a reactor and potassium carbonate was added (1.06 kg) followed by addition of ethanol (275 ml).

The reaction mixture was heated to 70-80° C. and benzyl chloride was added drop-wise (463 g) during a time period of 1 hour. Stirring was continued for 9 hours.

4 L of water was added to the reactor and the organic layer was washed for 15 minutes at temperature of 55-60° C., then mixing was stopped and the two layers were allowed to settle for additional 15 minutes. The layers were separated and additional 4 L of water was added to the organic layer and washing procedure was repeated for a second time followed by phase separation. A 47% solution of NaOH was added to the organic layer (470 ml) followed by ethanol (400 ml). The mixture was stirred at room temperature for one hour to afford a suspension.

Water (2 L) was added and stirring was continued for 15 minutes. The stirring was stopped to allow the two layers to settle for 15 minutes. The phases were separated and the organic layer was washed with water (2 L ml). The mixture was stirred at 25-30° C. and maleic acid (550 g) was added at same temperature.

Upon completion of the addition, the donepezil maleate salt precipitated from the two phase solution. Mixing was continued at 25° C. for 1 hour, and then mixture was cooled to 5-10° C. and mixing was continued at this temperature for 1 hour. The solid was obtained by centrifugation and the resulting cake was washed with cold water (2×1 L) followed by cold acetone (5.8 L) and dried. 1.39 Kg crude donepezil maleate was obtained in 90% yield having purity of 99.4% (by HPLC).

What is claimed is:

1. An improved process for alkylating secondary amines using an alkylating agent, wherein an alcohol serves as reaction facilitator.

2. The process according to claim 1, wherein the secondary amines are compounds having an amino piperidinic group of the type (IV), which are named: [4-(2-alkoxycarbonyl-5,6-dimethoxy-indan-1-on2-yl)-methyl]piperidine of the following general formula: 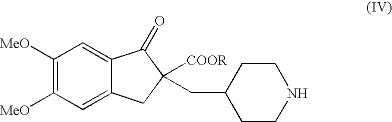
R═C₁-C₄ alkyl group or aralkyl group

3. The process according to claim 2, being used for obtaining 1-benzyl-4-[(5,6-dimethoxy-2-ethoxycarbonylindan-1-on-2-yl)methyl]piperidine by alkylation with an alkylating agent in an organic solvent, in presence of an inorganic base and an alcohol, the process comprising:
a) dissolving the compound of the type (IV), containing the amino piperidinic group, in an organic solvent and adding a base;
b) optionally adding an alcohol;
c) heating the reaction mixture, adding an alkylating agent optionally drop-wise and stirring for a time period sufficient to allow completing the reaction; and
d) isolating the product.

4. The process according to claim 3, wherein said alkylating agent is selected from the group consisting of benzyl bromide, benzyl chloride, and benzyl iodide.

5. The process according to claim 4, wherein the alkylating agent is benzyl chloride.

6. The process according to claim 3, wherein the organic solvent is selected from the group consisting of ethyl acetate, isopropyl acetate, n-butyl acetate, isobutyl acetate, dichloromethane, diethyl ether, diisopropyl ether, methyl tert-butyl ether, toluene, xylenes, and mixtures thereof.

7. The process according to claim 6, wherein the organic solvent is toluene.

8. The process according to claim 3, wherein the inorganic base is selected from the group consisting of sodium hydroxide, potassium hydroxide, sodium carbonate, sodium bicarbonate, potassium carbonate, potassium bicarbonate, and combinations thereof.

9. The process according to claim 8, wherein the inorganic base is potassium carbonate.

10. The process according to claim 3, wherein the alcohol is selected from the group consisting of methanol, ethanol, 1-propanol, 2-propanol, 1-butanol, 2-butanol, isobutanol, and mixtures thereof.

11. The process according to claim 10, wherein the alcohol is ethanol.

12. The process according to claim 11, wherein the percentage of the alcohol in the reaction mixture is 1-5% v/v relative to the volume of the organic solvent, preferably 1-3% v/v relative to the volume of said solvent and more preferably 2.5%.

13. The process according to claim 5, wherein at least 3.5 volume parts of benzyl chloride relative to 10 weight parts of said starting material are used in the reaction.

14. The process according to claim 5, wherein 4 volume parts ml of benzyl chloride relative to 10 weight parts of said starting material are used in the reaction.

15. 1-formyl-[4-[(5,6-dimethoxy-2-ethoxycarbonylindan-1-on-2-yl)methyl]piperidine of the formula:

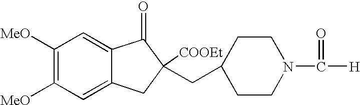

16. The compound 1-formyl-[4-[(5,6-dimethoxy-2-ethoxycarbonylindan-1-on-2-yl)methyl]piperidine of claim 15, being in substantialy pure form, and prepared by reacting [4-(5,6-dimethoxy-2-ethoxycarbonylindan-1-on-2-yl)methyl]piperidine and formic acid.

17. The use of a sample of the compound 1-formyl-[4-(5,6-dimethoxy-2-ethoxycarbonylindan-1-on-2-yl)methyl]piperidine of claim 15, as a reference marker in testing the purity of [4-(5,6-dimethoxy-2-ethoxycarbonylindan-1-on-2-yl)methyl]piperidine, obtained by hydrogenation of 1-CBZ-[4-(5,6-dimethoxy-2-ethoxycarbonylindan-1-on-2-yl)methyl]piperidine essentially as described herein.

18. An improved process, for deprotecting 1-CBZ-[4-(5,6-dimethoxy-2-ethoxycarbonylindan-1-on-2-yl)methyl]piperidine by hydrogenation while preventing the formation of the impurity 1-formyl-[4-(5,6-dimethoxy-2-ethoxycarbonylindan-1-on-2-yl)methyl]piperidine, using the sample of claim 17, the process comprising:
a) dissolving 1-CBZ-4-[(5,6-dimethoxy-2-ethoxycarbony lindan-1-on-2-yl)methyl]-piperidine in an organic solvent and adding a suitable amount of a catalyst;
b) hydrogenating optionally at elevated temperature and under pressure;
c) optionally filtering off the catalyst through Celite thus obtaining a solution;
d) evaporating the solvent under reduce pressure and without application of heat, or optionally with cooling, to prevent formation of impurities; and
e) isolating the product as a solid.

19. The process according to claim 18, wherein the preferable catalyst is 5%-10% palladium on charcoal, more preferably 5% palladium on charcoal.

20. The process according to claim 19, wherein at least 0.8 weight parts of palladium on charcoal relative to 10 weight parts of starting material are used in the reaction.

21. The process according to claim 20, wherein 1 weight part of palladium on charcoal relative to 10 weight parts of starting material are used in the reaction.

22. The process according to claim 18, wherein the product [4-(2-5,6-dimethoxy-ethoxycarbonyl-indan-1-on-2-yl)-methyl]piperidine is obtained in at least 95% yield and a having a purity of at least 98% (by HPLC).

23. A process for manufacturing Donepezil and the salts thereof in high yield and purity, using the processes or intermediates prepared and described herein.
